(19) United States
(12) Patent Application Publication
(10) Pub. No.: US 2020/0246605 A1
(43) Pub. Date: Aug. 6, 2020

(54) T-SHAPED CATHETER

(71) Applicants: Shelly SEGAL-FINKLER, Herzeliya Pituach (IL); Leonardo FERBER DRUMON, Belo Horizonte (BR)

(72) Inventors: Shelly SEGAL-FINKLER, Herzeliya Pituach (IL); Leonardo FERBER DRUMON, Belo Horizonte (BR)

(21) Appl. No.: 16/780,998

(22) Filed: Feb. 4, 2020

Related U.S. Application Data
(60) Provisional application No. 62/800,673, filed on Feb. 4, 2019.

(51) Int. Cl. A61M 39/02; A61M 1/36

(52) U.S. Cl. CPC A61M 39/0247; A61M 1/3661; A61M 2205/0266; A61M 2039/027; A61M 2039/0291; A61M 2039/0282; A61M 2039/0258

(57) ABSTRACT

The present invention provides a T- or Y-shaped catheter device for creating a permanent/long-term vascular access point for a patient in need of repetitive infusions/access or dialysis.

### FIELD OF THE INVENTION

The present invention relates to a catheter device, more particularly a T- or Y-shaped catheter device for creating a permanent/long-term vascular access point for a patient in need of repetitive infusions/access or dialysis.

### BACKGROUND

The development of hemodialysis for treating end-stage renal disease was followed by the development of various vascular access devices for acquiring and returning large quantities of blood for passage through an extracorporeal circuit during the hemodialysis procedure. Known devices usually rely on the use of either indwelling venous catheters or flow through shunt devices which create an artificial fistula between an artery and a vein.

Common venous catheters are limited since they provide relatively poor draw flow and irritate, which often results in vessel stenosis, thrombosis, and occasionally vessel perforation. Such catheters frequently fail due to infection, vessel wall weakness, poor positioning, and/or thrombus formation. As a result, shunt devices which create a fistulous blood flow between an artery and a vein have been developed. However, they have their own problems: installation is an extensive surgical procedure; once installed, the shunts require additional cardiac output (as much as one-fifth of the cardiac output); the transfer of the arterial pressure wave to the vein results in damage to the vein at the attachment point, which may lead to intimal hyperplasia and subsequent thrombosis and shunt occlusion; repeated punctures of the shunt's wall often result in eventual failure and require another surgery for repairing or replacing the shunt.

An alternative approach is the use of indwelling catheters which occupy only a portion of the vessel lumen, and are usually used for long term implantation in arteries. However, they are subject to movement within the vessel, resulting in irritation and vessel perforation. In addition, they disrupt normal blood flow in a manner which can damage the blood vessel and blood components, as well as encourage thrombosis.

Accordingly, a need exists for a catheter which causes minimal disruption of blood flow through the lumen, does not cause vessel stenosis, thrombosis, or vessel perforation, and is capable of handling large quantities of blood, and which will retain its usefulness for a long period of time after implantation.

Accordingly, various T-shaped catheters and cannulas have been developed aiming at solving the above and other disadvantages. Such T-shaped catheters are used for a variety of purposes, and are described, e.g., in U.S. Pat. Nos. 5,512,043; 5,443,497; 5,169,385; 5,041,101; 4,822,341; 4,639,247; 3,826,257; 4,421,507; and 3,516,408. However, these, and other known T-shaped catheters not mentioned herein have their disadvantages and shortcomings.

Therefore, there is an unmet need for developing new and simple to use catheter devices having a lateral port enabling easy and simple vascular access, which eliminate the need of complicated surgical procedures and repetitive puncturing of a patient's skin and blood vessels.

### SUMMARY

It has now been found, in accordance with the present invention, that the drawbacks of known catheter devices having a lateral port of the prior art, can be overcome by the presently claimed T-shaped catheter device.

Accordingly, the present invention provides a T- or Y-shaped vascular access device 100 for enabling access to a patient's vascular system, said device comprises: (a) an intraluminal tubular portion open at both ends 101,102, having an external diameter that is similar to the internal diameter of a blood vessel 200 into which it is to be dispositioned; (b) at least one stent 103, optionally tubular, interposed within said intraluminal tubular portion and extending outwardly from both of said open ends 101,102; and (c) an integral lateral access tubular portion 104, wherein said integral lateral access tubular portion 104 extends outwardly through or just beneath (subcutaneously) the skin of a patient after disposition of said vascular access device 100 within said blood vessel, such as to provide, when said vascular access device 100 is in place, an easy access/entry into said patient's vascular system, wherein said stent 103 is flexible or deformable such as to enable folding of said vascular access device 100 for inserting same into said blood vessel, and expansion thereof after insertion, and further enables fixation of both open ends 101,102 of said access device 100 inside said blood vessel, and wherein said integral lateral access tubular portion 104 prevents unintentional/undesired blood from exiting, while enabling access into the patient's vascular system.

The present invention further provides a system for enabling access to a patient's vascular system, said system comprising: (a) the T- or Y-shaped vascular access device 100 of the invention as described herein; (b) a port 300, for both securing the integral lateral access tubular portion 104 of said vascular access device 100 to the surface of the skin; and closing said device 100 for preventing contact between the outside and the blood system; and optionally (c) a guiding means designed for inserting and placing said device 100 in place within a patient's blood vessel.

The present invention further provides a method for creating a vascular access point in a patient in need thereof, said method comprising the steps of: (i) selecting a suitable area in a suitable blood vessel; (ii) determining the inner diameter of said blood vessel; (iii) selecting a device 100 of the invention having an adequate outer diameter; (iv) penetrating said blood vessel and inserting/positioning said device 100 therein; and (v) optionally fixing said device 100, thereby creating a vascular access point.

### BRIEF DESCRIPTION OF THE DRAWINGS

FIGS. 1A-1B are schematic illustrations of embodiments of T-shaped vascular access devices according to the invention.

FIG. 2 illustrates a vascular access device according to the invention in a pre-use state, with a balloon placed within.

FIGS. 3A-3C illustrate an insertion apparatus for inserting the vascular access device of the invention into the blood vessel of a patient. FIG. 3A is an illustration of an insertion apparatus used to pierce a blood vessel for placing therein a guiding sleeve; FIG. 3B illustrates how said insertion apparatus enters the blood vessel; and FIG. 3C illustrates how the guiding sleeve is left in the blood vessel.

FIGS. 4A-4F illustrate one possible way of inserting a vascular access device of the invention into a blood vessel of a patient.

FIGS. 5A-5B illustrate the valve within a vascular access device of the invention. FIG. 5A is a transparent view of the device; and FIG. 5B is a cut view.

### DETAILED DESCRIPTION

The present invention relates to subcutaneously implanted cannulas used to access the body's circulation. More particularly, the invention provides a T-shaped catheter and method for establishing intermittent vascular access using an implanted cannula in the general shape of a “T”.

Accordingly, the present invention provides a T- or Y-shaped vascular access device 100 for enabling access to a patient's vascular system, said device comprising: (1) an intraluminal tubular portion open at both ends 101,102, having an external diameter that is similar to the internal diameter of a blood vessel 200 into which it is to be dispositioned. In certain embodiments, said external diameter is at least 4 mm. In other embodiments, said external diameter is from about 4 to about 10 mm. However, it should be noted that the diameter may be less than 4 or more than 10, dependent on need and the patient's special blood vessel's diameter; (2) at least one stent 103, optionally tubular, interposed within said intraluminal tubular portion and extending outwardly from both of said open ends 101,102; and (3) an integral lateral access tubular portion 104, wherein said integral lateral access tubular portion 104 extends outwardly through or just beneath the skin (i.e. subcutaneous) of a patient after disposition of said vascular access device 100 within said blood vessel, such as to provide, when said vascular access device 100 is in place, an easy access/entry into said patient's vascular system, wherein said stent 103 is flexible or deformable such as to enable folding of said vascular access device 100 for inserting same into said blood vessel, and expansion thereof after insertion, and further enables fixation of both open ends 101,102 of said access device 100 inside said blood vessel, and wherein said integral lateral access tubular portion 104 prevents blood from exiting, while enabling access into the patient's vascular system.

In certain embodiments, the present invention provides a T- or Y-shaped short/long term vascular access device 100 for enabling access to a patient's vascular system, said device comprising: (a) an intraluminal tubular portion open at both ends 101,102, having an external diameter that is similar to the internal diameter of a blood vessel 200 into which it is to be inserted/dispositioned; (b) at least one stent 103, optionally tubular, interposed within said intraluminal tubular portion and extending outwardly from both of said open ends 101,102; (c) an integral lateral access tubular portion 104; and (d) a port 300 located-within or associated-with said integral lateral access tubular portion 104, wherein said integral lateral access tubular portion 104 extends outwardly through or just beneath the skin of a patient after disposition of said vascular access device 100 within said blood vessel, such as to provide, when said vascular access device 100 is in place, an easy access/entry into said patient's vascular system, wherein said stent 103 is flexible or deformable such as to enable folding of said vascular access device 100 for inserting same into said blood vessel, and expansion thereof after insertion, and further enables fixation of both open ends 101,102 of said access device 100 inside said blood vessel, and wherein said port 300 prevents blood from exiting the blood vessel freely, while enabling access into the patient's vascular system upon demand.

The vascular access device 100 according to the present invention may have various lengths, sizes (gouge) and shapes, depending on the designated use of such a device and the patient's vascular size. For instance, when intended to be used in an adult, a smaller gouge device is to be used, and when intended to be used in a child or infant, a higher gouge catheter is to be used. Exemplary gouge possibilities of the device 100 of the invention are 14 G, 16 G, 18 G, 20 G, 22 G, and 24 G. Notably, the vascular access device 100 of the invention may be made of flexible material which may be adjusted to fit a range of gouges, such that the stent(s) maintains its position in the patient's blood vessel after insertion.

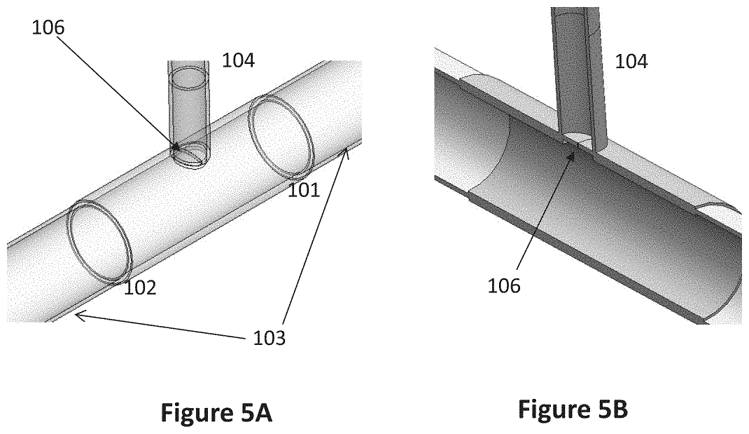

In certain embodiments, the vascular access device 100 of the invention further comprises a valve 106. In specific embodiments, said valve 106 is located at the connecting point between said lateral access tubular portion 104 and said intraluminal tubular portion, thereby enabling controlling the blood flow through said vascular access device 100. Alternatively, said valve 106 is designed to prevent blood from flowing upwardly into said lateral access tubular portion 104 (FIG. 5).

In other specific embodiments, said valve 106 is located horizontally at the tip of (or anywhere along) said lateral access tubular portion 104, enabling both preventing blood from exiting said blood vessel, as well as easy opening and closing said vascular access device 100 according to need, i.e. to withdraw blood and/or reintroduce blood and other fluids into the patient's vascular system.

In certain embodiments, the vascular access device 100 of the invention further comprises a port 300, said port 300 is designed to affix said integral lateral access tubular portion 104 in place and prevents its withdrawal into the body, as well as providing a simple access into the blood stream, e.g. by using a luer lock type connection.

In certain embodiments, the port 300 of the vascular access device 100 of the invention is designed to provide a simple access into the blood stream, e.g. by using a luer lock type connection, for either drawing blood therefrom or inserting blood and other fluids therein, as well as affix said integral lateral access tubular portion 104 in place and prevent its withdrawal into the body.

In certain embodiments, once the vascular access device 100 of the invention has been inserted into the patient's blood vessel, the outer walls of said intraluminal tubular portion are pressed against the inner walls of said blood vessel, thereby preventing blood flow therebetween and forcing the blood to flow only via said intraluminal tubular portion of said device 100.

In a specific embodiment, the vascular access device 100 of the invention has (i) a pre-use state, in which said device 100 is folded and said intraluminal tubular portion is flattened (i.e. has no free inner space); and (ii) an in-use state, in which said device 100 has been inserted into a patient's blood vessel, and is expanded such that the outer walls of said intraluminal tubular portion are pressed against the inner walls of said blood vessel, and there is an inner space within through which blood can flow.

In another specific embodiment, the vascular access device 100 of the invention has (i) a pre-use state, in which said device 100 is mounted onto a removable guiding needle designed to penetrate the patient's skin and blood vessel endothelium; and (ii) an in-use state, in which said device 100 has been inserted into a patient's blood vessel, and said removable guiding needle is removed, wherein upon removal of said removable guiding needle, said device 100 transforms from said pre-use state to the in-use state.

It should be noted that the term “transform” as used herein refers to the structural shift from a first form of the device 100 in which it is intended and designed to be inserted into a patient's blood vessel, into a second, open-active form, i.e. once the device 100 is in place. Such transformation may be obtained either automatically, e.g. due to material's memory (i.e. it strives to return to its original “open” form, or by manually widening/opening said intraluminal tubular portion, e.g. by widening/opening said at least one stent 103, by any suitable mechanism, e.g. a balloon 105 (one or more), a memory substance, electronically, etc.

In certain embodiments, the intraluminal tubular portion of the vascular access device 100 of the invention, has a front- and a rear-end, wherein once inserted into the patient's blood vessel, blood flows through said intraluminal tubular portion from said front-end towards said rear-end. In a specific embodiment, said intraluminal tubular portion further comprises inner one-way valve(s) mimicking the valves within blood vessels. In certain embodiments, said front- and rear-ends are identical. In other embodiments, said front- and rear-ends are different, e.g. in length, in width of the wall, in shape, in gouge size, etc. Said differences may be constructed in order to reduce blood flow interference and turbulences, reduce the risk of thrombosis and improve fixation of the device 100 to the blood vessel. In addition, said differences may be required due to the structure of each patient's blood vessel.

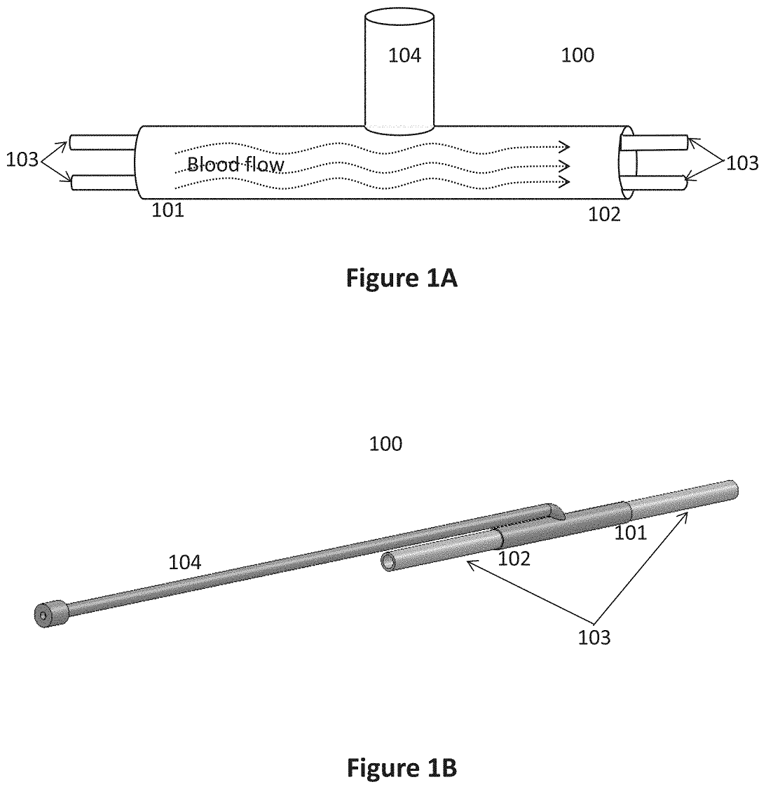

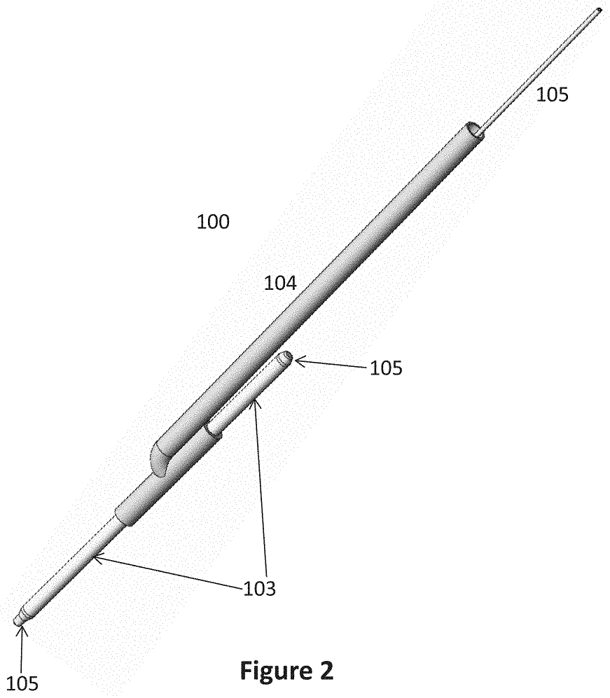

In certain embodiments, said at annular least one stent 103 of the vascular access device 100 of the invention, is designed according to need and may be in any shape and size. Accordingly, in certain embodiments, said at least one stent 103 is tubular, i.e. rounded and surrounds the entire inner surface of the open ends 101,102 of said intraluminal tubular portion (FIG. 2). Alternatively, said at least one stent 103 is not tubular, i.e. does not surround the entire inner surface of the open ends 101,102 of said intraluminal tubular portion, but is either arced- or rectangle-shaped, i.e. comprised of one, two, three, for or more extensions evenly distributed across the perimeter of the open ends 101,102 of said intraluminal tubular portion (FIG. 1).

In a specific embodiment, said at least one stent 103 is comprised of a single unit, extending from one open end 101 of said intraluminal tubular portion to the other end 102, and is either tubular or not. In yet another specific embodiment, said single stent is tubular except for the connection area of said lateral access tubular portion 104 with said intraluminal tubular portion. In yet another specific embodiment, said single stent is tubular inside said intraluminal tubular portion except for its connection area with said lateral access tubular portion 104, and is not tubular when extending from said open ends 101,102 of said intraluminal tubular portion.

In another specific embodiment, said at least one stent 103 is comprised of one, two, three, four or more units, extending from one open end 101 of said intraluminal tubular portion to the other end 102, wherein said units are is either arched- or rectangle-shaped, or a combination thereof.

In a specific embodiment, said at least one stent 103 constitute 1, 2, 3, 4, 5, 6 or more unassociated stent units.

In certain embodiments, said at least one stent 103 is positioned through the entire length of said intraluminal tubular portion. Alternatively, said at least one stent 103 is present only at the open ends 101,102 of said intraluminal tubular portion (and not through the entire length of said intraluminal tubular portion) and extending outwardly from both of said open ends 101,102.

In certain embodiments, said integral lateral access tubular portion 104 of the vascular access device 100 of the invention, is (i) opened for inserting a needle or tube therethrough or punctured with a needle so as to enable access into the patient's vascular system, and (ii) re-sealed upon extracting said needle or tube, so as to prevent blood from exiting. In a specific embodiment, the vascular access device 100 of the invention further comprises a designated seal mounted at the top open end of said integral lateral access tubular portion 104, which is used for closing said integral lateral access tubular portion 104 once treatment is concluded and no vascular access is further needed.

In specific embodiments, said integral lateral access tubular portion 104 is designed to adhere/wrap-around said inserted needle or tube thus preventing blood from exiting/escaping therebetween, e.g. is made of flexible memory material such as rubber or silicon.

In another specific embodiment, said integral lateral access tubular portion 104 is affixed in place by the aid of a designated port 300 that both fixes said integral lateral access tubular portion 104 in place and prevents its withdrawal into the body, as well as providing a simple access into the blood stream, e.g. by using a luer lock type connection. In yet another specific embodiment, said port 300 also constitutes as a seal or comprises a seal.

In certain embodiments, said intraluminal tubular portion and said lateral access tubular portion 104 of the vascular access device 100 of the invention, are made of a bio-graded flexible material. In specific embodiments, said bio-graded flexible material is silicon or rubber, or a combination thereof.

In other embodiments, said stent 103 of the vascular access device 100 of the invention, is made of a rigid bio-graded material, such as bare metal, nitinol, plastic, polycarbonate, cobalt chrome, or a combination thereof.

The vascular access device 100 of the invention and the stent 103 within may be made of any suitable material, preferably medical graded material. The term “medical graded material” as used herein refers to any biocompatible material, rigid or flexible, which can be sterilized by any suitable procedure/technique. In certain embodiments, the device of the invention is made of silicon or rubber, or any combination thereon; and the stent 103 is made of stainless steel, e.g., stainless steel 304, 304L, 316 or 316L; titanium or a titanium alloy, e.g., Ti-6Al-7Nb or nickel-titanium (nitinol); plastic; polyethylene; silicon; or rubber, or any combination thereon.

In certain embodiments, the present invention further provides a system for enabling access to a patient's vascular system, said system comprises: (i) the T- or Y-shaped vascular access device 100 of the invention; and (ii) a port 300, for both securing the integral lateral access tubular portion 104 of said vascular access device 100 to the surface of the skin; and closing said device 100 for preventing contact between the outside and the blood system.

In a specific embodiment, the system according to the invention further comprises (iii) a guiding means designed for inserting and placing said device 100 in place within a patient's blood vessel. In a specific embodiment, said guiding means is disposable. In another specific embodiment, said guiding means is re-usable and is sterilized prior to each use. In one embodiment, said guiding means comprises a piercing needle 201 used to penetrate the blood vessel 200 and a guiding sleeve 202 through which said device 100 is inserted into the blood vessel 200, and optionally an insertor pin 203 used to push said device 100 in.

In certain embodiments, after production of the vascular access device 100 or system of the invention it is preferably packed in a sealed and sterilized package until use.

In certain embodiments, the device or system of the invention can be used in the treatment of dialysis or peripheral vascular disease, or any other disease or condition that requires repetitive access to a patient's vascular system.

The present invention, in other embodiments, also provides a method for creating a vascular access point in a patient in need thereof, said method comprising the steps of: (i) selecting a suitable area in a suitable blood vessel; (ii) determining the inner diameter of said blood vessel; (iii) selecting a device 100 of the invention having an adequate outer diameter [i.e. fitting said inner diameter of said blood vessel]; (iv) penetrating said blood vessel and inserting/positioning said device 100 therein; and (v) optionally fixing said device 100, thereby creating a vascular access point.

In certain embodiments, the method of the invention further comprises an additional measuring step after inserting/positioning said device 100 in said blood vessel, for determining the actual diameter of the vessel, and performing necessary adjustments. Non-limiting examples of such necessary adjustments include replacing the device 100 with a better suited device, changing the device's insertion location or its anchoring point, etc. or any combination thereof.

In a specific embodiment, the step of fixing said device 100 in place according to the method of the invention is carried out by securing the integral lateral access tubular portion 104 of said vascular access device 100 to the surface of the patient's skin.

In another specific embodiment, the step of fixing said device 100 in place according to the method of the invention is carried out by attaching said port 300 to the integral lateral access tubular portion 104 of said vascular access device 100 and then securing said port 300 to the body of the patient, e.g. securing same to the surface of the patient's skin.

In yet another specific embodiment of the method of the invention, the step of fixing said device 100 in place within said blood vessel is carried out via said stent(s) 103 which is attached-to or merged-with the inner walls of the blood vessel after expansion thereof.

Specific, non-limiting, embodiments of the invention will now be illustrated with reference to the accompanying figures.

FIG. 1 describes two possible configurations of a T-shaped vascular access device 100 of the invention. FIG. 1A illustrates a T-shaped vascular access device 100 having two stents 103 extending from each end 101,102 of its intraluminal tubular portion. FIG. 1B illustrates a Y-shaped vascular access device 100 having a single tubular stent extending from each end 101,102 of its intraluminal tubular portion. It should be noted that the device illustrated in FIG. 1B may also be considered as a T-shaped device. In either case, the integral lateral access tubular portion 104 is either flexible enough to enable its bending over as illustrated in the figures, or comprises a dedicated flexible connection area enabling it free bending and moving.

FIG. 2 the same vascular access device 100 as shown in FIG. 1B, this time with an uninflated balloon 105 placed therein. This is an illustration of the device 100 in a pre-use state, i.e. before placed inside a blood vessel and before inflating said balloon 105 for extending said stent(s) 103.

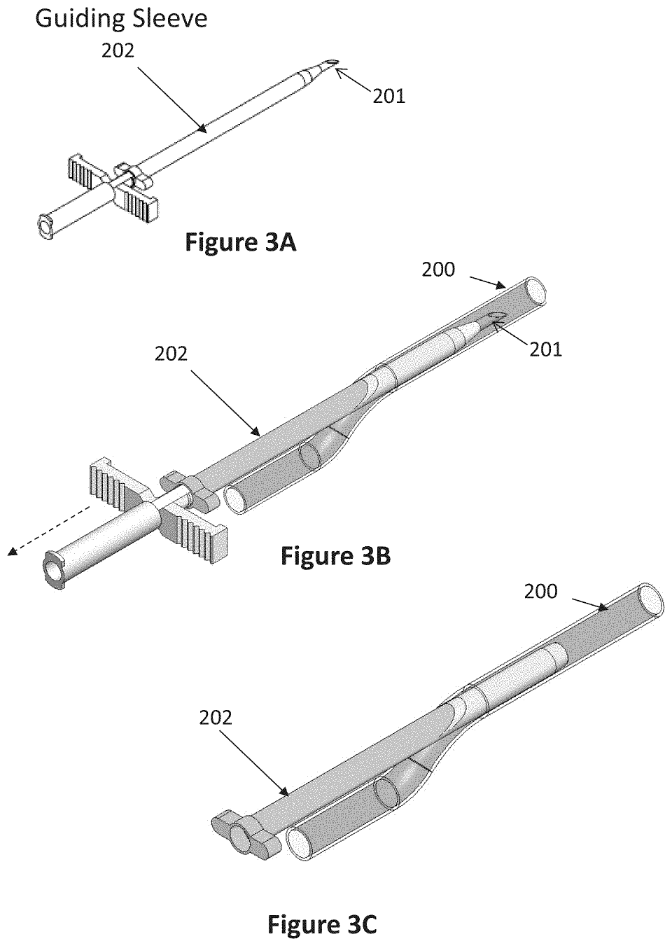

FIGS. 3A-3C illustrate one possible configuration of an insertion apparatus suitable for the insertion of the vascular access device 100 of the invention into a blood vessel 200 of a patient. FIG. 3A illustrates said insertion apparatus having a removable piercing needle 201 positioned within a guiding sleeve 202. As illustrated in FIG. 3B, after piercing the blood vessel 200, said removable piercing needle 201 is pulled out to be removed, leaving behind only said guiding sleeve 202 (FIG. 3C) positioned in the blood vessel 200 and extending outwardly from the patient's body, thereby enabling the physician to insert the device 100 into the blood vessel 200.

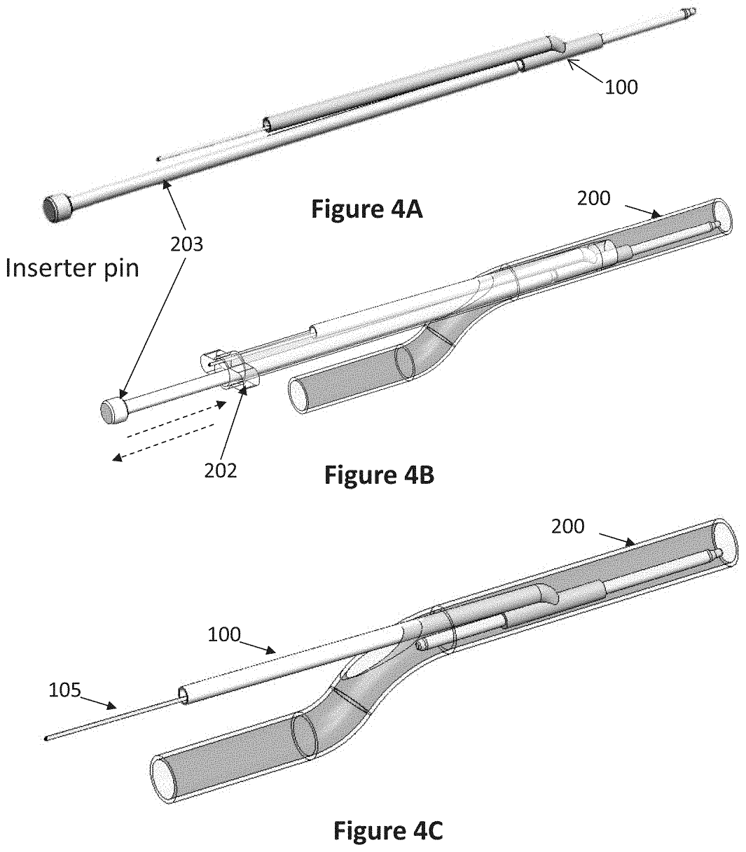

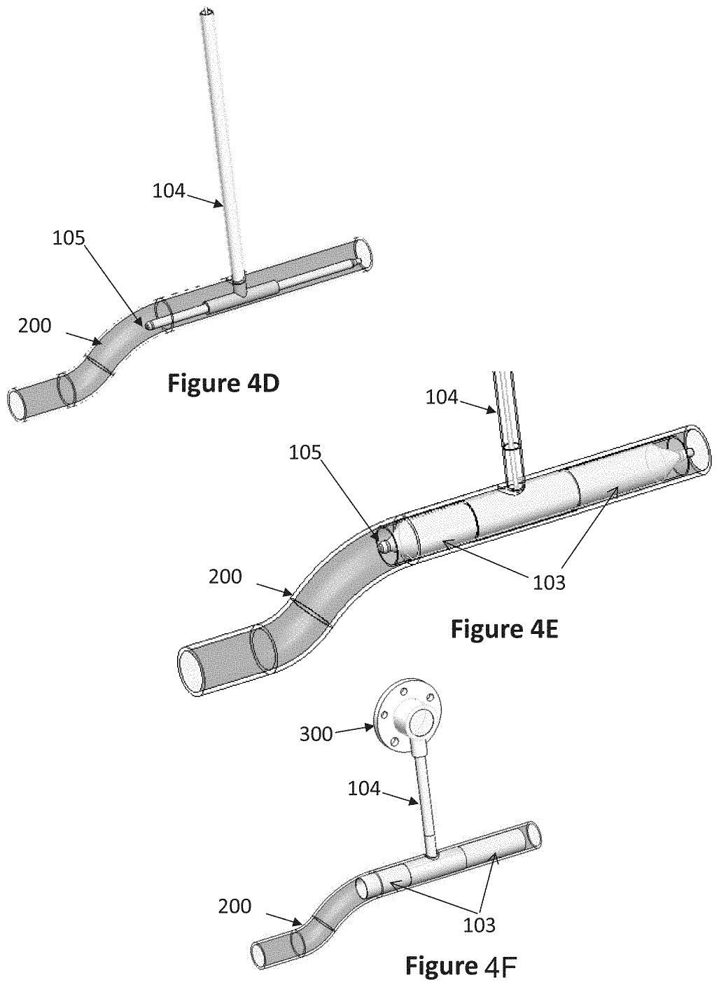

FIGS. 4A-4F illustrate one possible way of inserting a vascular access device 100 of the invention into a blood vessel 200 of a patient according to one embodiment of the method of the invention: After inserting a guiding sleeve 202 into the patient's blood vessel 200, e.g., as illustrated in FIGS. 3A-3C, a vascular access device 100 of the invention, in a pre-use state and mounted-on or associated-with an inserter pin 203 (FIG. 4A) can then be pushed through said guiding sleeve 202 and into the blood vessel 200 (FIG. 4B). The device 100 is pushed in until the proximal side of the stent 103 passes the blood vessel's insertion point—this can be determined, e.g., by any suitable imaging or fluoroscopic technique, by feel, etc. After insertion, both the guiding sleeve 202 and inserter pin 203 are removed/retrieved, leaving behind only the vascular access device 100, still in its pre-use state (FIG. 4C). In certain embodiments, the integral lateral access tubular portion 104 may require being slightly pulled in order to better positioning the device 100 prior to fixation and anchoring said the integral lateral access tubular portion 104 in place (FIG. 4D). Next, the device 100 is transformed into its in-use state, e.g. by inflating a balloon 105 located therein. The balloon 105 extends and “opens” both the intraluminal tubular portion and said stent(s) 103 interposed within said intraluminal tubular portion and extending outwardly from its open ends (FIG. 4E). After the device 100 has transformed into its in-use state, the balloon 105, or any other mechanism which was used to transform the device 100 from its pre-use state to its in-use state, is removed (in the case of a balloon 105—it is first depleted for ease of removal). Now, the integral lateral access tubular portion 104 is anchored, e.g. using said port 300, to the patient's skin (FIG. 4F), thereby preventing the withdrawal of said integral lateral access tubular portion 104 into the body, while providing an easy vascular access as well as preventing blood from exiting. In certain embodiments, said integral lateral access tubular portion 104 is first cut to a desired length before connecting same to aid port 300.

FIGS. 5A-5B illustrate how a valve 106 may be located within a vascular access device 100 of the invention. Such a valve may be either a one-way valve allowing only the insertion of fluids therethrough, or a self-sealing valve that can re-seal itself after opening/penetrating, e.g. with a needle or tube.

1. A T- or Y-shaped short/long term vascular access device 100 for enabling access to a patient's vascular system, said device comprising:
a. an intraluminal tubular portion open at both ends 101,102, having an external diameter that is similar to the internal diameter of a blood vessel 200 into which it is to be dispositioned;
b. at least one stent 103, optionally tubular, interposed within said intraluminal tubular portion and extending outwardly from both of said open ends 101,102;
c. an integral lateral access tubular portion 104; and
d. a port 300 located within said integral lateral access tubular portion 104,
wherein said integral lateral access tubular portion 104 extends outwardly through or just beneath the skin of a patient after disposition of said vascular access device 100 within said blood vessel, such as to provide, when said vascular access device 100 is in place, an easy access/entry into said patient's vascular system,
wherein said stent 103 is flexible or deformable such as to enable folding of said vascular access device 100 for inserting same into said blood vessel, and expansion thereof after insertion, and further enables fixation of both open ends 101,102 of said access device 100 inside said blood vessel, and
wherein said port 300 prevents blood from exiting the blood vessel freely, while
enabling access or withdrawal into the patient's vascular system upon demand.

2. The vascular access device 100 of claim 1, further comprising a valve 106.

3. The vascular access device 100 of claim 1, wherein said port 300 is designed to provide a simple access into the blood stream, as well as affix said integral lateral access tubular portion 104 in place and prevents its withdrawal into the body.

4. The vascular access device 100 of claim 1, wherein once inserted into the patient's blood vessel, the outer walls of said intraluminal tubular portion are pressed against the inner walls of said blood vessel, thereby enabling the blood to flow only via said device 100.

5. The vascular access device 100 of claim 1 having (i) a pre-use state, in which said device 100 is folded and said intraluminal tubular portion is flattened (i.e. has no free inner space); and (ii) an in-use state, in which said device 100 has been inserted into a patient's blood vessel, and is expanded such that the outer walls of said intraluminal tubular portion are pressed against the inner walls of said blood vessel.

6. The vascular access device 100 of claim 1 having (i) a pre-use state, in which said device 100 is mounted onto a removable guiding needle designed to penetrate the patient's skin and blood vessel endothelium; and (ii) an in-use state, in which said device 100 has been inserted into a patient's blood vessel, and said removable guiding needle is removed, wherein upon removal of said removable guiding needle, said device 100 transforms from said pre-use state to the in-use state.

7. The vascular access device 100 of claim 1, wherein said at least one stent 103 is tubular.

8. The vascular access device 100 of claim 1, wherein said at least one stent 103 is not tubular, and is arced shaped or rectangle-shaped.

9. The vascular access device 100 of claim 8, wherein said at least one stent 103 constitute 1, 2, 3, 4, 5, 6 or more unassociated stent units.

10. The vascular access device 100 of claim 1, wherein said at least one stent 103 is positioned through the entire length of said intraluminal tubular portion.

11. The vascular access device 100 of claim 1, wherein said at least one stent 103 is present only at the open ends of said intraluminal tubular portion and extending outwardly from both of said open ends 101,102.

12. The vascular access device 100 of claim 1, wherein said integral lateral access tubular portion 104 is (i) opened for inserting a needle or tube therethrough or punctured with a needle so as to enable access into the patient's vascular system, and (ii) re-sealed upon extracting said needle or tube, so as to prevent blood from exiting.

13. The catheter device of claim 1, wherein said stent 103 is made of a rigid bio-graded material, such as bare metal, nitinol, plastic, polycarbonate.

14. A system for enabling access to a patient's vascular system, said system comprising:
a) the T- or Y-shaped vascular access device 100 of claim 1; and
b) a port 300, for both securing the integral lateral access tubular portion 104 of said vascular access device 100 to the surface of the skin; and closing said device 100 for preventing contact between the outside and the blood system.

15. The system of claim 16 for enabling access to a patient's vascular system, said system further comprising:
c) a guiding means designed for inserting and placing said device 100 in place within a patient's blood vessel.

16. A method for creating a vascular access point in a patient in need thereof, said method comprising the steps of:
selecting a suitable area in a suitable blood vessel;
determining the inner diameter of said blood vessel;
selecting a device 100 according to claim 1 having an adequate outer diameter;
penetrating said blood vessel and inserting/positioning said device 100 therein;
and
optionally fixing said device 100,
thereby creating a vascular access point.

17. The method of claim 18 further comprising an additional measuring step after inserting/positioning said device 100 in said blood vessel, for determining the actual diameter of the vessel, and performing necessary adjustments.

18. The method of claim 18, wherein said fixing of said device 100 in place is carried out by securing the integral lateral access tubular portion 104 of said vascular access device 100 to the surface of the skin.

19. The method of claim 18, wherein said fixing of said device 100 in place is carried out by attaching a port 300 to the integral lateral access tubular portion 104 of said vascular access device 100, thereby securing same to the surface of the skin.

20. The method of claim 18, wherein said fixing of said device 100 in place is carried out by the stent(s) 103 attaching-to/merging-with the inner walls of the blood vessel.
